(19) United States
(12) Patent Application Publication
(10) Pub. No.: US 2006/0275555 A1
(43) Pub. Date: Dec. 7, 2006

(54) Painting method and apparatus

(76) Inventor: Daniele Colizza, Rapallo (GE) (IT)

Correspondence Address: Serafini Associates, 7660 FAY AVE. STE H378, LA JOLLA, CA, 92037, US

(21) Appl. No.: 10/553,106

(22) PCT Filed: Feb. 10, 2004
(86) PCT No.: PCT/EP04/50104

§ 371 Date: Dec. 20, 2005

(51) Int. Cl. A61M 11/00; B05C 5/00; B05D 1/04

(52) U.S. Cl. 427/458; 118/313; 128/200.23; 132/200; 128/200.14; 118/620

(57) ABSTRACT

A method and apparatus for painting animals and/or objects, using a coating paint product, characterized in that it has: at least one column-like upright member at least one spray nozzle fitted on said column at least one tank containing said paint liquid an electrically insulated, shower tray-like platform at least a jet-containing wall, which is likely to be situated in front of said column-like upright member.

The invention relates to a method and apparatus for painting animals and/or objects, using a coating paint product.

Particularly, the invention relates to an apparatus used for coating solid bodies with coating paint liquid compounds, which are vaporized and/or micronized and/or atomized while spraying a body to be coated, in accordance with a novel and particularly convenient and cost effective method.

The practice of coating bodies with more or less liquid substances has been known since ancient times, which practice is carried out by a massage action with a number of various methods, obviously including the manual action.

The main drawbacks arising from this practice are based on the fact that the manual massage action is particularly difficult if the expected degree of coating is particularly high, e.g. when a liquid, a cream, a lotion or a general compound having tanning properties is to be applied, in which case the body on which said compounds are applied shall not only exhibit a degree of coating next to 100%, but also and further a particularly even and homogeneous coating, due to the fact that said coating shall allow the skin that is treated with the above compounds to react to sun exposure, or to react according to the chemical properties of the compounds and substances in use. This is the case of sunless tanning creams and lotions, by which certain structural components of the skin react chemically by color variations (like in the case of ammines and certain small polypeptides which take a golden to dark brown color depending on the amount of compound being applied). The skin is composed of various cells having different characteristics, which can give it important characteristics to be considered in the design and optimization of an apparatus for coating it. The skin has a multifaceted surface having both a lipophilic and lipophobic character, which allow the skin to function as a kind of barrier against unwanted impurities, and yet to let the gaseous component of the atmosphere in and water vapor or liquids out One more factor to be considered in the design and, optimization of a coating , paint product application apparatus is that due to the particular texture of the skin, a full and evenly homogeneous coating is hardly attainable. It is also known that, in order to fully and homogeneously coat a body, one more person is often needed, especially for coating regions of the body that are hardly reachable by the subject (e.g. back, shoulders) and skin areas at joints, in whose folds, often due to the massage movement, the liquid and/or cream in use stagnates.

Apparatuses and methods are known in the art for spraying electrostatically charged liquid paints, to enhance the adhesion of the coating paint solution or liquid on the skin of the subject, which obviate many of the drawbacks caused by manually coating a body with liquids and/or creams, particularly having tanning properties. In certain particular embodiments, closed booths are used, in which coating paint liquids are sprayed on subjects in said booths. From prior art various types of closed booths are known for painting with coating paint products having various useful characteristics, for a proper application of the product on the body of the subject, such as the use of thermal fans to keep the atmosphere inside the painting booth warm and comfortable, and/or the possibility of ventilating the environment (booth) at the end of the painting cycle, thereby obtaining an obvious improvement of the environment conditions, in view of a further painting cycle on another subject and/or the presence of air-cleaning systems, which clean the air inside the booth by using fans, which are operated at each painting cycle and remove by suction the liquid paint mist component fluctuating in the air during the cycle, which might be inhaled by the subject that is subjected to the painting action.

As most prior art apparatuses comprise closed booths, they cause considerable drawbacks during use. Some of these drawbacks are of psychological nature, as it is known that some people react with irritability and general discomfort in certain situations, for instance when they are forced to be enclosed in a rather small space (as a painting booth might be), thereby causing a sort of claustrophobia even in people who do not generally suffer from this pathology.

Another drawback that may arise from the use of prior art apparatuses is the possibility that the treated subject may receive a slight electric shock when the subject comes even in slight contact with the outside of the paint liquid or solution spray nozzles, as said electrostatic charge induced in the coating paint liquid or solution to enhance the adhesion of the coating paint solution or liquid is induced at the spray nozzles.

It was surprisingly found that many of the technical and not technical drawbacks of prior art apparatuses may be easily obviated, thereby drastically improving the quality of the service provided by said apparatuses and reducing costs both of manufacture and use of said apparatuses.

This invention has the object of obviating these drawbacks by a simple, cost-effective and easy to implement arrangement, which is also more interesting for the subject that is expected to use said invention, by providing a painting booth of the open type, which is made of at least two walls disposed one in front of the other and has one or more paint liquid or solution spray nozzles through which said liquid is propelled under pressure, and exits therefrom in a finely micronized and/or atomized and/or vaporized form, and used various methods to charge electrostatically the solution and/or liquid being used, to achieve the purpose of fully and homogeneously coat the body.

According to an improvement, said painting booth is conformed in such a manner, as to form a sort of shower cabin, which is composed of at least two walls, preferably made of a transparent material and placed one in front of the other in a discontinuous manner, so as to leave at least two areas uncovered by said walls, which are particularly suitable to allow the movements of the incoming and ingoing subjects to be exposed to painting cycles.

It may be noted that an arrangement involving the construction of painting booths formed by two opposed walls, as described above is particularly interesting in that it allows to obviate certain typical drawbacks of prior art closed booths, as this particular arrangement allows people who may not feel at ease in particularly narrow environments to use these apparatuses for the application of lotions and/or solutions all over the body, as it considerably decreases the tension generated by the fear of closed places, thanks to the fact that the subject may easily put an end to a painting session by simply going out of the booth with no need of opening a door, which makes the booth much more comfortable.

Furthermore, such booths may be simply used on beaches and other public spaces as they need no particular maintenance, unlike closed booths.

A further improvement consists in that said booths need no particular for cleaning and ventilate the environment like those in use for closed booths, because the natural aeration of the environment (booth) limits the stagnation of the coating paint liquid aerosol inside the environment, and the booth wall washing process is further facilitated by the openings on the sides of the walls and by the presence of a rotating platform, provided as an easily removable and cleanable tray, which is highly useful for possibly recycling the coating paint solution in use during the painting cycle or for a faster disposal of the exhausted component of said solution, as there is no pipe connection through which said solution would be forced to flow.

An additional advantage is provided by the different methods used, according to the invention, to charge electrostatically the liquid in use during the painting process.

As mentioned above, the use of electrostatically charged liquids is known in the process of animal and/or object painting, with the aim of optimizing the adhesion of the micronized and/or atomized and/or vaporized liquid on the body being painted.

The above well-known apparatuses use spray nozzles which induce the electrostatic charge on the coating paint liquid to be used in the painting step, while said liquid exits from the nozzles and, as discussed above, may have the drawback of retaining an electric charge, though minimal, which might be discharged on the user of the apparatus, if he/she moves inside the closed booth.

A further advantage provided by the apparatus of this invention consists in that it provides various methods for inducing an electrostatic charge on the coating paint liquid.

In a particular embodiment of the invention, a tank is used which can induce the electrostatic charge on the solution contained therein by using one or more spark plugs and/or diodes and/or electrodes which are directly in contact with the liquid or dipped therein, thereby preventing the electrostatic charge from being induced at the spray nozzles, and consequently preventing any passage of electrostatic current from the nozzles to the body of the subject being painted, due to a contact between the subject and the charged nozzles.

A further embodiment uses micronizing and/or atomizing and/or vaporizing nozzles inside a polarization further containing electrodes and/or diodes and/or spark plugs, which come in direct contact with the vaporized mist of the liquid used for the painting process and directly induce the electrostatic charge thereon by contact.

In a further embodiment of the inventive apparatus the liquid is passed through a transparent coil, which is wound around or in contact with at least one UV lamp. UV rays are known to be able to induce an electrostatic charge on a liquid passing near it, without any direct contact with electrostatically charged metal elements, and thereby preventing any short circuit condition.

In a further embodiment, micronizing and/or vaporizing and/or atomizing nozzles are provided in a polarization chamber wherein micronization and/or vaporization. and/or atomization occur until liquid droplets of about 20 μm are formed, which droplets are naturally charged with a positive electrostatic charge due to the high pressure exerted thereon.

Further improvements or characteristics of the painting method and apparatus forming the subject of the dependent claims.

These and other characteristics and advantages of the invention will appear more clearly from the following description of a few embodiments shown in the accompanying drawings, in which:

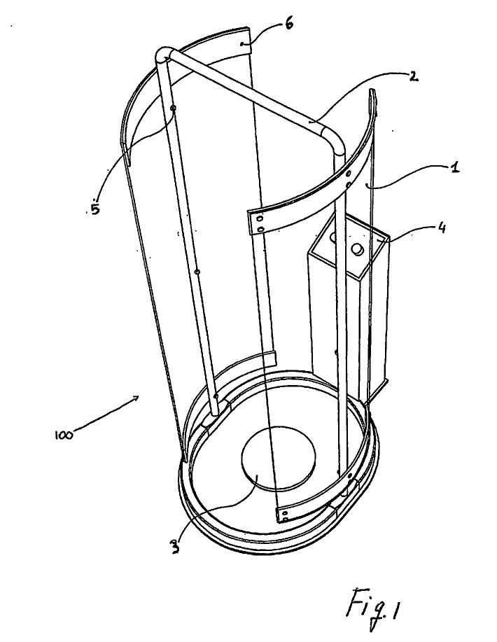

FIG. 1 is a top and side perspective view of a painting booth.

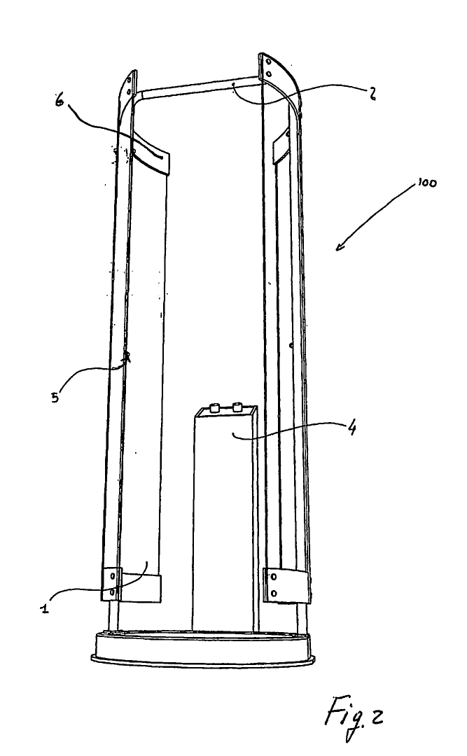

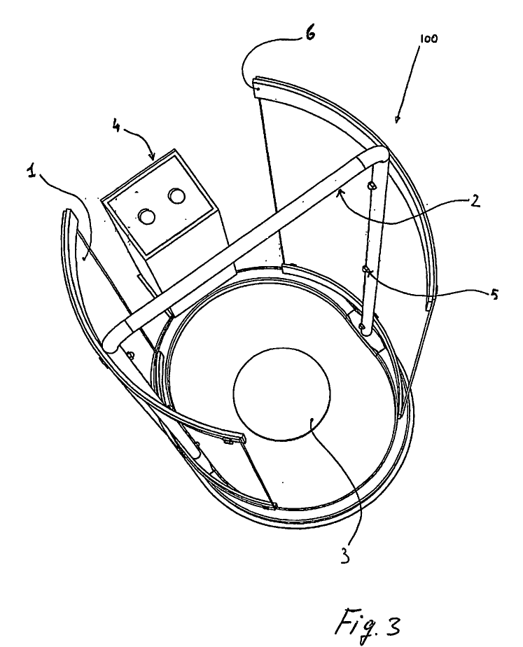

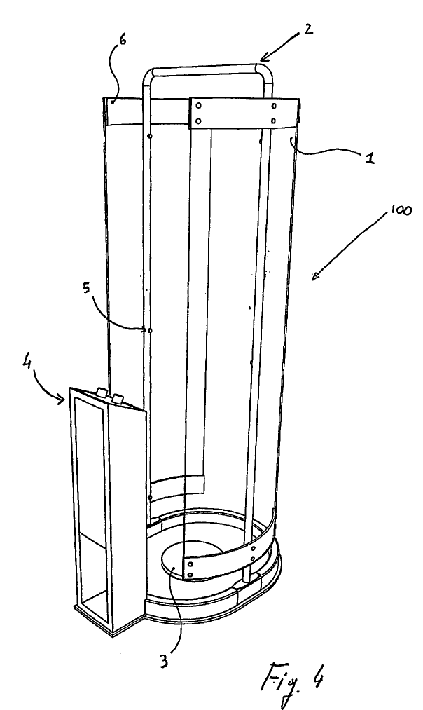

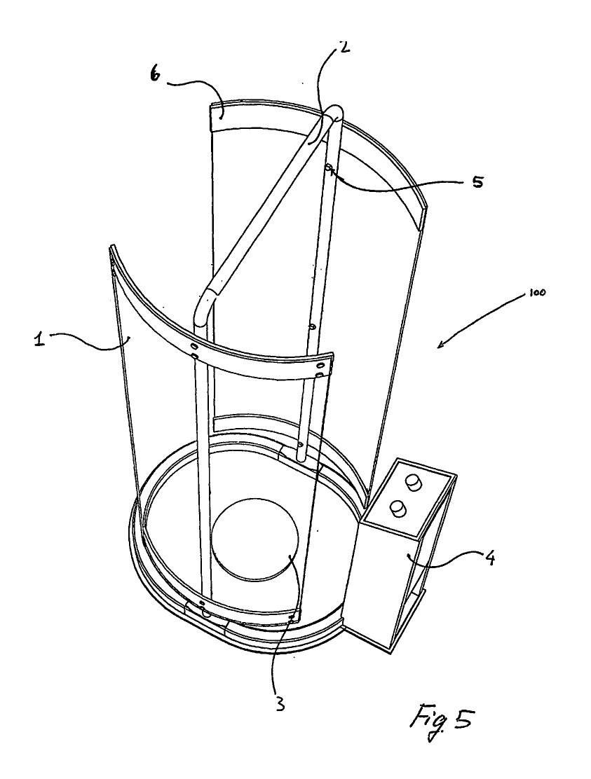

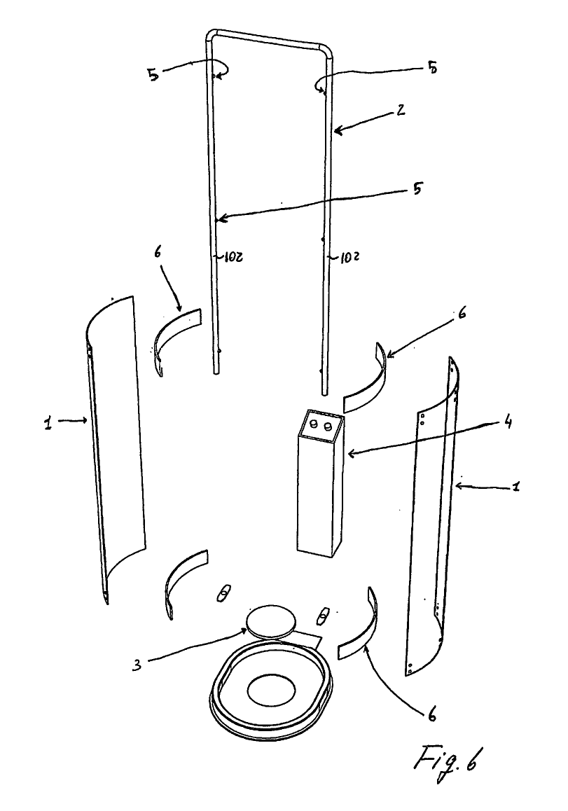

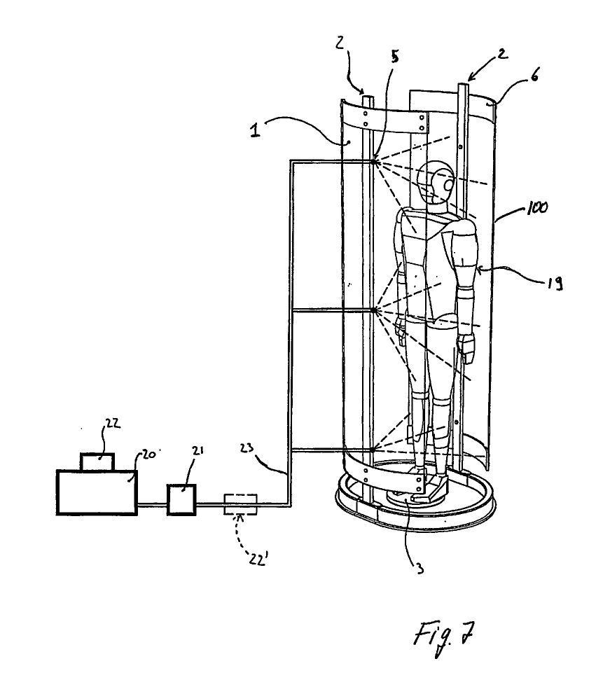

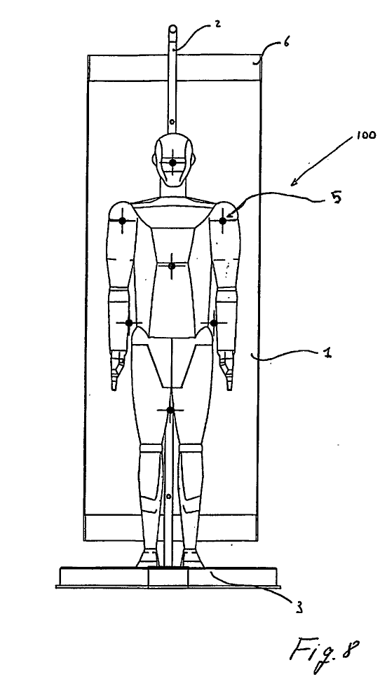

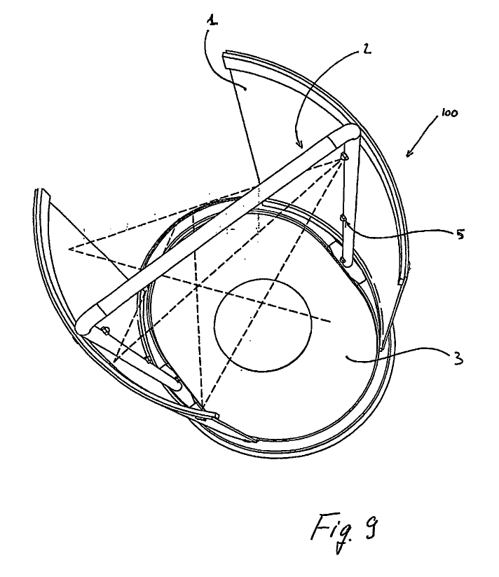

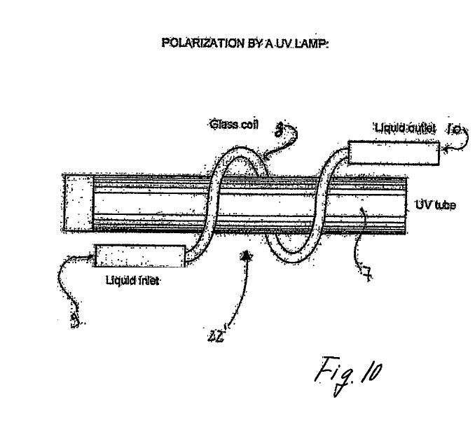

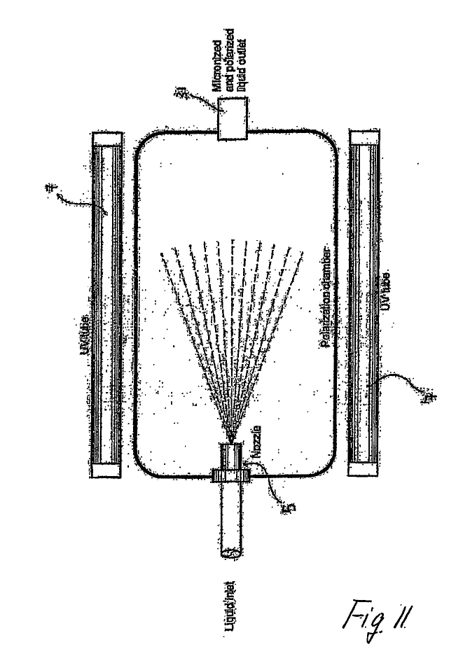

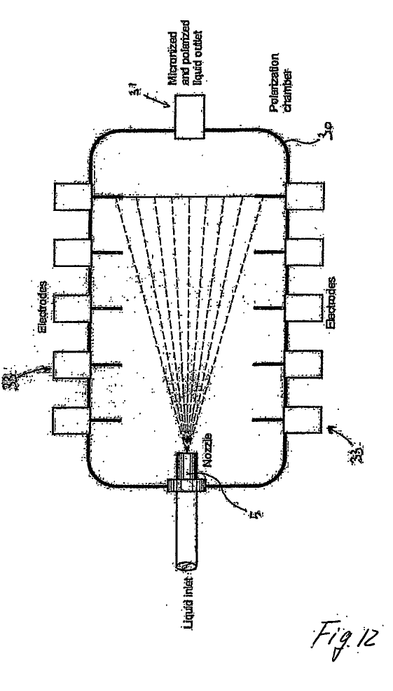

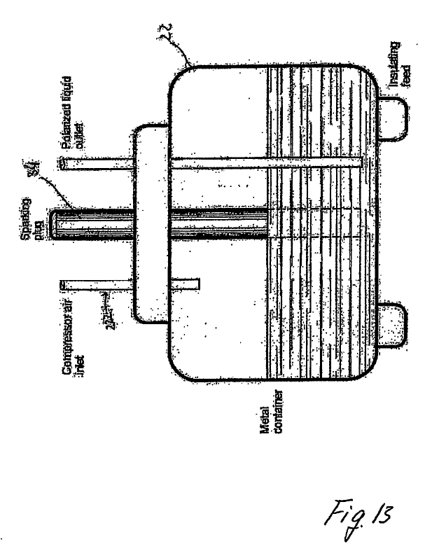

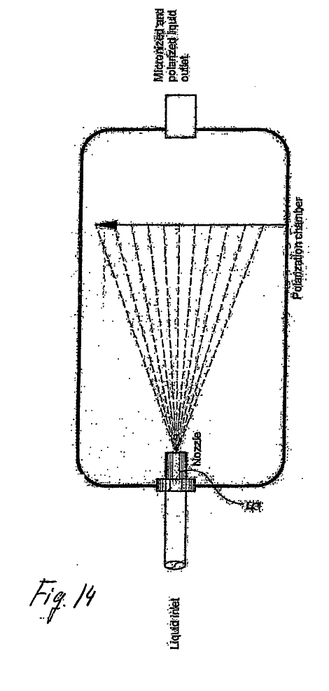

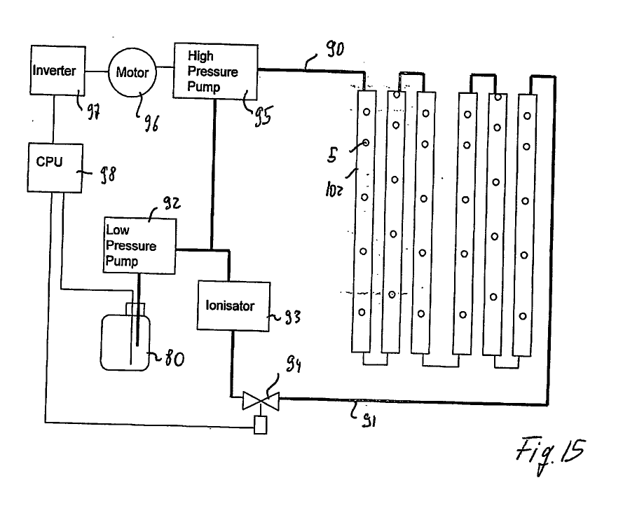

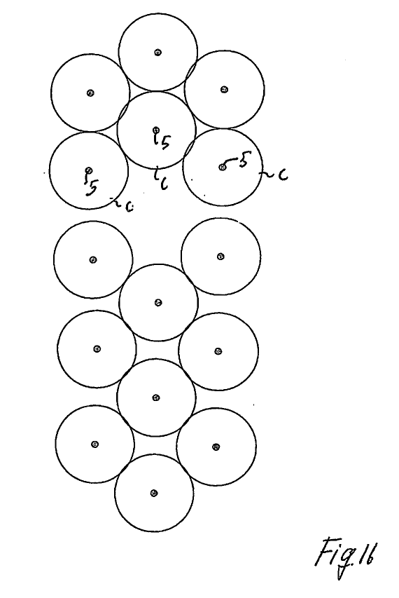

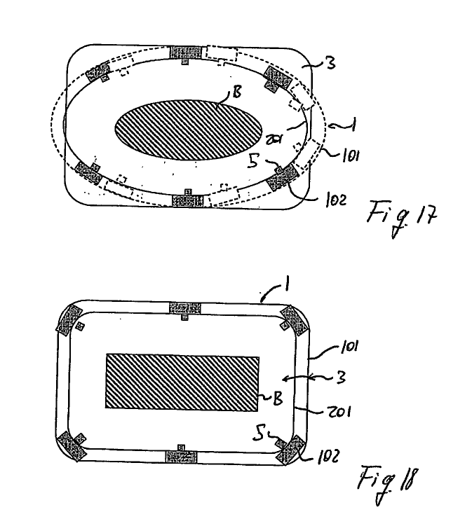

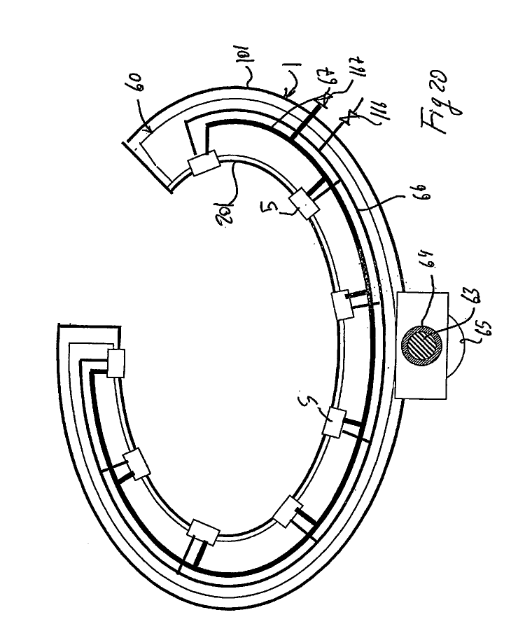

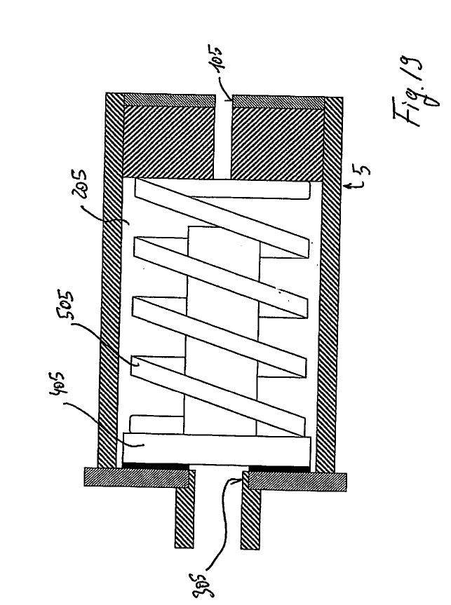

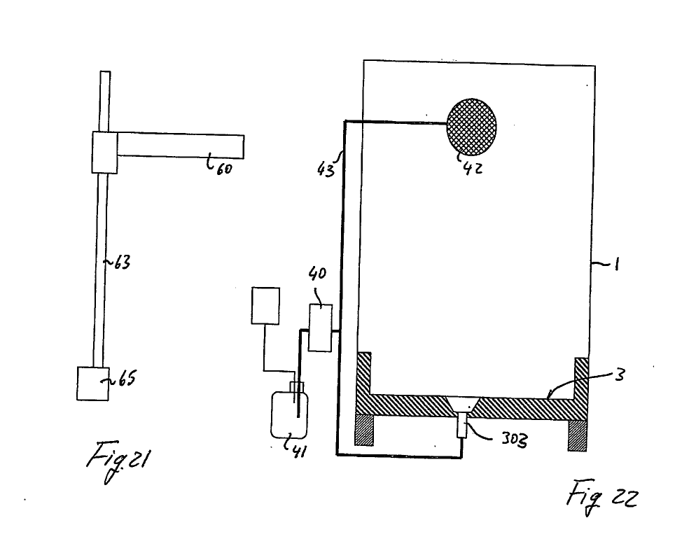

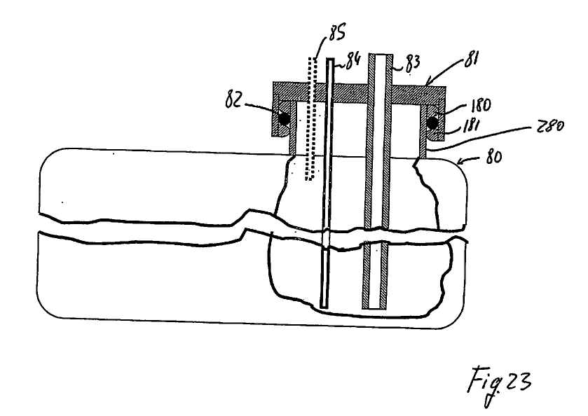

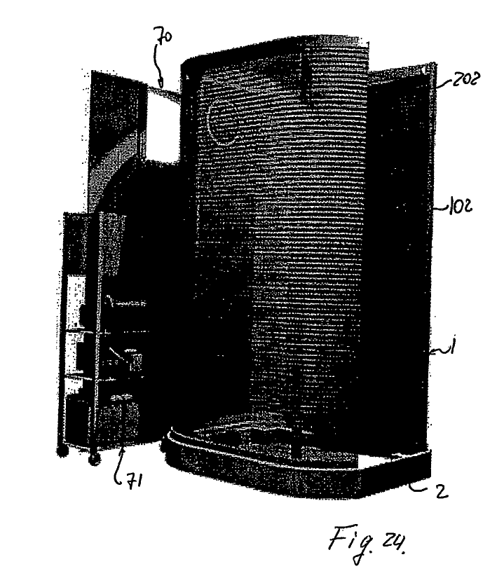

FIG. 2 is a front and side perspective view of a painting booth.

FIG. 3 is a top view of a painting booth.

FIG. 4 is a rear and side perspective view of a painting booth.

FIG. 5 is a top and side perspective view of a painting booth.

FIG. 6 shows the individual construction elements of the painting booth.

FIG. 7 is a side perspective view of the shower with a subject in it, showing a block diagram of the circuit along which the material to be sprayed is delivered.

FIG. 8 is a diagram of the subject body locations hit by the spray.

FIG. 9 is a spray atomization pattern within the curvilinear walls of the booth.

FIG. 10 shows a paint liquid polarization scheme by using a UV lamp.

FIG. 11 shows a liquid polarization scheme in a polarization chamber by using two UV lamps.

FIG. 12 shows a liquid polarization scheme of the liquid in a polarization chamber by using electrodes.

FIG. 13 shows a liquid polarization scheme in a metal tank. FIG. 14 shows a liquid polarization scheme after 20 μm spraying.

FIG. 15 shows a further embodiment of the apparatus according to the invention.

FIG. 16 shows the disposition of the spraying nozzles for obtaining optimum distribution of the tanning liquid over the body of a user.

FIGS. 17 and 18, show respectively a schematic view from above of the booth according to the invention and the position of the columns for forwarding the liquid to the nozzles and which carry the said nozzles.

FIG. 19 shows a cross-sectional view along an axial plane of a preferred embodiment of the nozzles.

FIG. 20 shows another embodiment of the spaying nit of the device according to the invention the other art of the device being omitted except the double lateral wall.

FIG. 21 shows a lateral view of the embodiment according to FIG. 20.

FIG. 22 show a simplified schematic cross-sectional view of the device according to the invention in which vent means for sucking away the rests of the sprayed liquid and a bottom wall with means for collecting and transporting away the fall down rests of the sprayed liquid are provided.

FIG. 23 shows a schematic view of the tank for the tanning liquid or the like according to a further variant of the present invention.

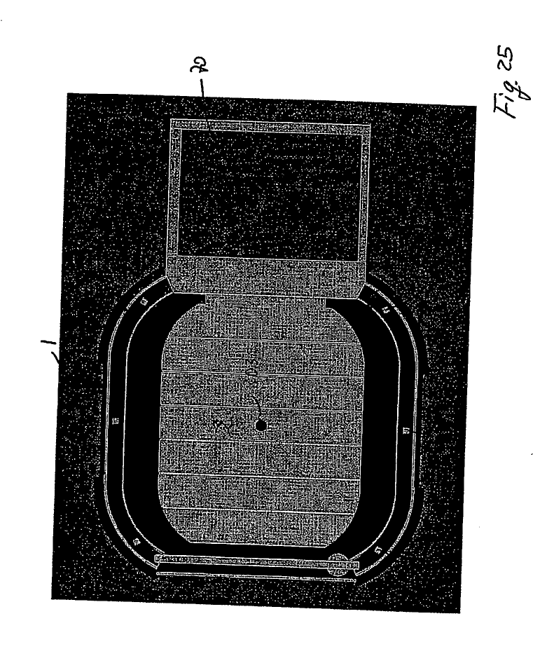

FIG. 24 shows a perspective view of a further embodiment of the device according to the invention.

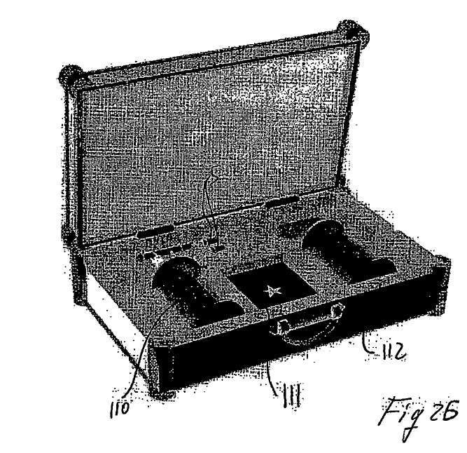

FIG. 25 shows a top view of the embodiment according to FIG. 24.

FIG. 26 show a schematic view of a device according to the invention in a portable form.

FIGS. 1 to 5 are top and side perspective views of a painting booth. The booth 100 has two curvilinear walls 1 which are connected by an upright 2 having nozzles, a rotating tray-like platform, a controller element 4, spray nozzles 5 and elements for supporting the walls 6. Note that the construction of the above booth is particularly suitable for people who might suffer from particular disturbances related to the difficulty of finding themselves in closed and narrow spaces, and that the particular shape of the curvilinear walls, which are inwardly concave, is particularly useful in keeping the vaporization and/or atomization and/or micronization mist generated by the spray nozzles 5 as long as possible within the environment (booth). The tray, walls and column may be made of any material of various values, depending on aesthetic needs and/or other needs arising from installation sites. Particularly, the tray or at least a portion thereof is made of an electrically conductive material and is connected to the ground potential by known and appropriate means.

FIG. 6 shows the individual construction elements of the painting booth as shown in the previous figures, but also highlights the booth construction simplicity, which simplicity increases both the cost-effectiveness of the process, no particular technical arrangement being, required for maintenance, and the particular versatility of the apparatus, that may be used in a variety of contexts, such as in a gym or a beach. A preferred embodiment includes a tubular inverted U-shaped upright 2 having nozzles in one or more of its stems, and providing the additional function of supporting the curvilinear walls.

FIG. 7 schematically shows the booth according to the previous figures, as well as a block diagram of the paint liquid spray circuit.

The paint liquid is drawn from a tank 20 and pressurized by using a pump 21. Means 22 for electrostatically charging the paint liquid may be associated to or inserted in the tank 20, which means will be described in greater detail hereafter. Alternately, as outlined in dashed lines, said means 22′ may be of such a type as to generate a radiative ionization and are provided in the pipe 23 which delivers the liquid to the spray nozzle/s 5. The pipe 23 may serve one, two or more spray nozzles 5, which are fitted on the column 2 in various patterns and/or orientations relative to the spraying direction. When more than one spray nozzle 5 is provided, a manifold, not shown in detail, may be advantageously provided, the individual pipes for connection to the nozzles 5 branching off it.

Referring to FIG. 8, spray nozzles 5 may be arranged on the column 2 in such a pattern as to ensure an optimized distribution of the paint liquid over the user's body. Considering an average body size, the spray nozzles 5 are advantageously arranged in a particular pattern, providing a nozzle substantially level with the face, two nozzles level with the shoulders, each coinciding with one of the two shoulders, a nozzle level with the chest, particularly the sternum, two nozzles, each coinciding with the end of the forearm, particularly in the wrist region, assuming a vertical downward position of the arms, and a nozzle in a central position between the knees. Position adjustment means may be provided for each nozzle relative to the others, along both the vertical and the horizontal axes. Hence, a few adjustments and simple removable nozzle supporting means may be sufficient to adjust and adapt the position of the nozzles to the various sizes of the user's bodies, while maintaining said optimized arrangement of nozzles.

In accordance with an additional advantageous characteristic of the inventive booth, the relative position of the column, the tray and the curvilinear wall opposite the column is such that the body of the patient is situated substantially at the focus of the curvilinear wall with reference to the spray direction of the nozzles, therefore even when a fixed platform is provided, the part of the body opposite the column is painted in an optimized, substantially even manner. In FIG. 9, this is shown by dashed lines, which outline the main spraying direction of the nozzles and the corresponding reflection direction. Obviously, such characteristic largely depends on the overall geometry of the booth, hence the relative positions of the above mentioned parts as well as the nozzle direction and the curvature of the wall may greatly vary, provided that the body of the patient, hence the tray, are always kept substantially at the focus of the curvilinear wall.

Regarding the means 22, 22′ for electrostatically charging the paint liquid, these may be substantially of two types.

A first embodiment, as shown in FIG. 10, provides a radiative ionization by using an UV lamp, designated as 7. Near the UV lamp 7, the nozzle feeding pipe is made of a transparent glass portion 8 which is advantageously shaped as a coil wound around a UV lamp 7 having the shape of a tube or a cylinder. Said combination lamp and coil is provided as a separate radiative ionization unit 22 and has a liquid inlet 9 and a liquid outlet 10. The advantages of this method for electrostatically charging the liquid that passes through the coil 8 consist in that there is no need for direct contact between the liquid and an electric conductor, for said liquid to acquire the desired electrostatic charge, which provides obvious advantages in terms of operational safety, as it prevents any short circuit danger mainly arising from the fact that these liquids contain a high percentage of water, which is highly conductive, as well as any potential direct connection between the user and an electric source.

FIG. 11 shows an alternative to the arrangement of FIG. 7. Here the radiative ionization unit is not provided in the nozzle feeding circuit, but is a part of a unit that is associated to or integrated in the nozzle. Each nozzle 5 opens into a ionization chamber 30, having an outlet for the micronized or atomized liquid, designated with numeral 31, directly opposite the nozzle. The nozzle sprays the liquid in the ionization chamber 30, whereto one or more UV lamps 7 are associated. Advantageously, the lamps are placed outside the ionization chamber 30 which is properly made of material that is transparent to UV rays. This allows to prevent any electric connection of the lamps which may come in direct contact with the paint liquid.

FIG. 12 shows a liquid polarization scheme in a ionization chamber, wherein electric electrodes are provided in direct contact with the atomized or nebulized liquid. Here, the ionization chamber 30 is fitted with one or more ionization electrodes 33, which may be simple electrodes made of copper or another electrically conductive material, or ionization diodes. Particularly, two rows of plate-like electrodes are disposed on two opposite sides of the ionization chambers 30, extending in opposite directions in a comb-like arrangement. Ionization may occur by applying a potential difference between the two rows of electrodes, or by charging the electrodes to a predetermined electrostatic potential, so that they may charge electrostatically the vaporized liquid.

FIG. 13 shows a variant for electrostatic charging of the paint liquid. Here, electrostatic charging occurs in the tank 20 by means of a ionization electrode, plug or diode 34 which is dipped in the liquid contained in the tank 22. Also, FIG. 13 shows an alternative liquid pressure feeding arrangement, which uses compressed air. In this case, the outlet of a compressor is connected to an inlet 222 of the tank and exerts a pressure on the liquid contained in the tank, which pressure propels the liquid into the outlet pipe 122.

Finally, according to FIG. 14 the sprayed liquid is ionized by the mechanical atomization action. It was found that, by micronizing the liquid to a droplet size of about 20 micron, the liquid so micronized had an electrostatic charge, probably due to the polar character of the liquid, which increases considerably at such small sizes. This mechanical arrangement is particularly advantageous as it does not require separate or additional means for ionization or electrostatic charging of the liquid to be sprayed, the process being a side effect of atomization/misting.

As appears clearly form FIGS. 1 to 9, the device according to the invention is formed by a cabin, or the like having lateral walls 1 and a bottom formed by a tray-like platform 3.

In the embodiments according to FIGS. 17, 18, 20, 22, 24 e 25 different variants of the booth described above are shown.

From the constructive point of view relatively to the booth or cabin itself lateral walls 1 and a tray like bottom platform 3 is provided. The lateral walls can be formed by a double layer wall having an outside wall layer 101 and an inner wall layer 201 which form a hollow space between each other. In this space the uprights pipes 102 carrying the nozzles 5 are provided.

The pipes 102 are connected together by a connection or distribution pipe 202. The tray like platform 3 has a concave shape in order to form a basin. The basin has preferably a wall degrading towards a collecting output 303 which is connected to an output pump 40 which forwards the fall down rests of the spayed liquid to a collecting tank 41. A pipe 43 connects the output pump 40 to a suction vent 42 which has condenser means as for example a centrifugal drier device (not illustrated) for condensing the liquid still dispersed in the atmosphere inside the booth, which is then forwarded by the output pump to the collecting tank 41.

As it appears clearly form FIGS. 17, 18, 24 and 25, four or more upright pipes 102 are provided, each one of which carries one or more nozzles 5. The uprights 102 are placed in the hollow space between the two lateral wall sheets 101, 201, the internal wall sheet having apertures coinciding with each nozzle 5.

FIGS. 17, 18, and 25 show a preferred positioning of the upright pipes which allow to have the nozzles carried by each upright pipe 102 essentially at a similar distance to the side of the body of the user against which the nozzles 5 are oriented.

The position of the uprights can follow two principal solids approximating the shape or enveloping surface of a human body.

According to FIG. 17 the human body is approximated by an ellipsoid or a regular ellipse. Thus the uprights pipes 102 carrying the nozzles are arranged along the surface of a solid having an elliptic basis being said upright pipes 102 oriented parallel to the axis of the solid, namely perpendicular to the elliptic basis.

The sheets 101, 201 forming the lateral walls are concentric solids having elliptic basis. Six upright pipes are distributed on opposite sides of the elliptic basis in a symmetric way relatively to the largest diameter of the elliptic basis. The distribution may be also be non symmetric. In FIG. 17 the body is indicated by B.

Another solid for approximating the shape of the human body is a parallelepipedon. A cross section thereof is indicated with B in FIG. 18. Here the six upright pipes 102 are placed at the centre of the longest laterals side and at each angle of the rectangular basis of the paralellepipedon formed by the bottom 3 and by the lateral wall 1

Obviously the examples of FIGS. 17 and 18 are schematic ones and do not take care of the dimension of the body and of the booth for allowing to provide an opening which is sufficient to easy enter the booth.

FIG. 25 is a view in which the principle of FIG. 17 or 18 is applied and in which correct dimensions are chosen for allowing to provide a sufficient large entrance in the booth.

As it appears from FIG. 17 further upright pipes 50 can be provided having also nozzles 51 for spraying for example air and or water, either for treating a user or for cleaning the booth after a spray treatment of the tanning liquid or the like. The said further uprights pipes 50 and associated nozzles 51 are illustrated with discontinuous lines. And are place on one side of the upright pipes 102 carrying the tanning liquid. Further upright pipes for spraying or distributing other liquids or gases may be provided at other positions. As it appears clearly also these further upright pipes 50 are provided in the hollow space between the two sheets 101, 201 forming the lateral walls and the inner sheet 201 is provided with apertures for the nozzles 51.

In another constructive variant illustrated in FIGS. 20 and 21, no upright pipes are provided, but a series of nozzles are carried on the interior surface of a bracket 60. These bracket is shaped as an open ring having an elliptic or circular or else shaped form. The form can be also a polygonal or square or rectangular one. The nozzles 5 are connected to a pipe 61 forwarding the liquid to be spayed.

The bracket is supported in a cantilevered way by a vertical sliding guide and means are provided for displacing the bracket up and down in a continuous way. Different means can be used which are within the choice of the expert in the art between the known ones. In FIG. 20 the guide and driving means are formed by a vertical threaded bar 63 which engages an internally threaded sleeve 64 which is mounted in a non rotatable way within a radial extension of the bracket 60.

A motor 65 drives the bar 63 in order to rotate around its axis. Other guiding and driving means can be sued such as electro-pneumatic actuators or similar.

The bracket 60 is housed within the hollow space formed between the two sheets 101, 201 forming the said wall. In this embodiment the internal layer has vertical axial openings which are continuous along the entire path of the corresponding nozzle 5.

As a further variant the embodiment of FIGS. 20 and 21 shows a different kind of nozzles for vaporizing and or atomizing the liquid to be sprayed. In this case the nozzles 5 uses a atomizing or vaporizing vector which is formed by air under pressure. These can be mixed to the liquid to be sprayed in a tank not shown and or as illustrated in FIG. 20 the air under pressure is mixed up with the liquid directly in the nozzles 5. As it is shown in FIG. 20 each nozzle is fed with the liquid and with the air under pressure by two different feeding lines indicated with 66 and 67.

These embodiment has the advantage that by providing valves at the inlet of each one of the two feeding lines as indicated by 167 and 166 only air or only liquid can be fed to the nozzles, thus allowing to carry out different functions by the same nozzles as for examples cleaning function by feeding only water to the nozzles and/or drying functions by feeding only air to the nozzles.

A further advantage of the embodiment according to FIG. 20 and 21 consist in the fact that the bracket can be displaced with different speeds at different heights in order to rest more time at the heights of anatomically difficult parts relatively to the spraying of the tanning liquid such as for example the female breasts.

According to FIGS. 24 and 25 the booth according to the invention may show a room for housing all the pumps, tanks and electronic units needed to operate the device. These units may be arranged on a trolley which can be pushed inside or drawn outside the housing room indicated with 70. These room 70 has a door and the different pipes or cables connection the devices on the trolley 71 with the units at the booth are formed by flexible pipes and cables and have a sufficient length to allow the drawing out of the trolley form the housing room 70.

FIG. 23 shows a further variant of the invention. According to this variant no stationary or dedicated tank is provided in the apparatus, but the usual bottles are used in which the tanning liquid or the like is sold, a bottle cap being provided having clamping means which provides for a mechanical retention and for generating the necessary seal. Generally the said bottles 80 have an annular flange 180 at a cylindrical output sleeve 280 which communicates with the interior of the bottle 80. The closure cap 81 according to the invention is provided with an internal annular flange 181 which can be elastically forced over the external flange 180 of the sleeve 208. The internal flange 181 is provided at a position of a lateral cylindrical wall of the cap at which position the said internal flange 181 is axially spaced from the external flange 180 on the sleeve 280 when the cap is fitted on the sleeve 280, the said distance forming an annular housing for an annular seal 82 such an 0-ring or the like. At least a suction pipe 83 passes through the cap and reaches the bottom of the bottle. Also a liquid level sensor 84 may be mounted in the cap for sensing the minimum level of the liquid in the bottle. Both the suction pipe 83 and the liquid level sensor 84 are sealed within the cap.

As it appears also from FIG. 23, the same or similar cap can be used in combination of a tank for collecting the rests of the prayed liquid sucked or retrieved from the booth after a treatment. I this case the collecting tank can be formed by an empty bottle 80. On the contrary of the cap for the full bottles when the empty bottle is used to collect the rests of the sprayed liquid from the booth, then a maximum level sensor is provided in the cap. This sensor indicated with 85 is illustrated with discontinuous lines in FIG. 23.

The same pipe 83 can serve as output pipe for extracting the liquid form the bottle 80 or as a feeding pipe for feeding the rests of the sprayed liquid collected from the booth after the treatment.

The maximum level sensor 85 and the minimum level sensor 84 can be of the kind generating an electric signal which is fed to a central control unit which controls the entire operative units of the apparatus, thus allowing to stop the apparatus when the bottle 80 from which the liquid to be sprayed reaches the minimum level and/or when the bottle in which the liquid resting in the booth after treatment is collected reaches the maximum level. Since the said bottles are of plastic material the above disclosed arrangement allows to spare plastic material with economic advantages and with a reduced pollution effect.

Although the caps are described with reference to a particularly shaped bottle 80, it is obvious for the expert in the art to modify the said cap in order to fit differently shaped bottles particularly relatively to the sleeve 280 so to achieve the same functions and advantages as the disclosed example.

According to still another embodiment of the apparatus of the present invention, FIG. 15 illustrates a modified liquid forwarding, ionisating and spraying circuit.

In this embodiment, the nozzles 5 are mounted on upright pipes 102. This is not necessary at all since the circuit can operate also with nozzles mounted on a bracket 60 according to FIG. 20.

The feeding circuit of the liquid to be sprayed to the nozzles 5 comprises a feeding branch 90 and a return branch 91. A pump sucks the liquid from a tank, particularly from the bottle 80 into the circuit 90, 91. In the return branch 91 a ionisation apparatus 93 for the circulating liquid is provided. A valve 94 is provided for closing the return branch 91 in order to generate the pressure in the feeding branch which allows praying. Furthermore valves are provided which closes the nozzles 5 during circulation when the valve at the return branch 91 is open.

Advantageously the circuit is provided with a pump furnishing a adjustable feeding pressure for the liquid, while the nozzles are provided with automatic pressure sensitive valves which opens when the liquid pressure overcomes a certain predetermined minimum pressure.

Advantageously the circuit is provided with two pumps. A first pump 92 which is a low pressure pump. The low pressure pump 92 feeds the liquid from the bottle or from the tank 80 into the circuit 90, 91. The pressure of the liquid when the valve 94 in the return branch 91 is open and the low pressure pump 92 is driven is below the minimum pressure level needed to operate the nozzles valves in order to commutate the said nozzle valves into the opened condition.

The low pressure pump 92 causes the liquid to circulate in the branches 91 and 90 and through the ionisation apparatus 93 for a period of time needed to ionisate the entire liquid circulating.

In the feeding branch 90 a high pressure pump 95 is provided through which the liquid can circulate when driven in the circuit 90, 91 by the low pressure pump 92. Typically a piston pump does not create to much resistance to the liquid flow when it is not operated.

The high pressure pump 95 is driven by a brushless motor 96 which is controlled by an inverter 97. A CPU unit 98 controls the entire process of the low pressure pump 92, of the inverter 97, the electrovalves 94 and the liquid level in the battle 80.

Ionisation can also be measured by means of electric voltage measurements or by simply measuring the time of the ionisation phase.

When the ionisation phase is ended the high pressure pump 95 is activated and the valve 94 in the return branch 91 is closed. Also the low pressure pump is closed and the pressure increases in branch 90 and at the nozzles 5 till the said pressure overcomes the minimum pressure for opening the nozzle valves allowing the liquid to be sprayed.

The circuit for the liquid is dimensioned in order to contain several liters of the fluid so that a great amount of fluid is ionised. Each treatment of spraying the liquid uses from 100 to 800 ml of liquid so that the liquid in the circuit is sufficient for several treatments.

Nevertheless a treatment can be advantageously splitted into several short spraying phases, at each spraying phase only a partial amount of the entire volume of liquid being sprayed. After each spraying phase the high pressure pump can be disactivated and the low pressure pump 92 is activated by opening again the valve 94 in the return branch for starting a ionisation cycle as disclosed above. After the ionisation cycle a new partial spray cycle can be carried out.

FIG. 19 illustrates a nozzle 5 being provided with a automatic pressure sensitive valve.

The nozzle 5 has a spraying opening 105 is mounted at an end of a feeding chamber 205. The feeding chamber 205 has an in out opening 305 opposite to the nozzle opening 105 the said opening forms a valve seat at the internal face with which a piston like valve shutter 405 cooperates. The said piston like valve shutter 405 is able to slide away and against the valve seat at the input opening 305 and is urged against the said valve seat by means of an elastic element 505. The elastic element 505 for example a spiral spring as illustrated in FIG. 19 is placed between a wall of the chamber 205 on the side of the nozzle opening 105 and opposite to a contact surface with the piston like valve shutter 405. The elastic element 505 is dimensioned in such a way as to need a certain predetermined input pressure of the fluid for allowing the piston like valve shutter 405 to be displaced away from the valve seat allowing the fluid to fill the feeding chamber 205 and reach the nozzle opening 105.

The said nozzle valves has the further advantages that they can be set to a fluid pressure near the optimum pressure allowing the liquid to be sprayed only when the pressure of the liquid is at a pressure near the optimum one. This avoid the fact that when the pressure is to low the nozzles may not sufficiently vaporize or atomize the liquid and to big drops or jets are emitted by the nozzles.

According to another feature of the invention, which is illustrated in FIG. 15 and 16, the nozzles on each upright pipe 102 are not positioned all at equal distances one from the other. Particulalrly the nozzles at the level of the breast are positioned closer to each other. Furthermore the nozzles on the upright pipes 5 on one side and on the other side of the largest diameter of the ideal path along which the upright pipes are positioned (see FIG. 17 and 18) are positioned axially displaced. The nozzles 5 on one upright pipe 102 are axially displaced relatively to the nozzles of the adjacent one. This allows to avoid overlapping of the praying comes of the nozzles ensuring maximum coverage.

FIG. 16 illustrates the nozzles 5 on three adjacent upright pipes (not shown) and the relative spraying cones at a distance at which a body is placed from the said nozzles. The bigger circles C indicates the cones on a flat surface at the said distance from the nozzles.

From the technical point of view the high pressure pump is drive in order to reach a spraying pressure from 30 to 100 bars, preferably from 60 to 70 bar. Each spraying cycle lasts several seconds particularly from 1 to 3 seconds.

The nozzles and the pressure of the liquid are defined in such a way as to generate drops having a range between 5 and 40 microns and to avoid turbulences.

Providing several shorts spraying cycles which are alternate to rest periods, allows to exploit better the enveloping effect of the polarized sprayed liquid and the natural falling down effect due to gravity.

Preferably a device according to figure 15 has the following operative cycle:

The liquid from the tank is sucked into the circuit by means of the low pressure pump 92 which is a self triggering one.

First a stand-by pahes is carried out during which the low pressure pump 92 sucks the liquid and continue to make it circulate in the circuit so that the liquid is continuously mixed and continuously passes through the ionisation device.

A ready to start phase during which the high pressure pump is activated at a low level rotation speed so that the pressure does not increase as high as to open the nozzle valves but the pressure in the system reaches a higher pressure from which the optimum spraying pressure can be rapidly reached.

A spraying phase in which the high pressure pump is brought to a higher rotation rate such as to allow the spraying pressure to be reached. The electro valve in the return branch 91 are closed and the pressure increases at the level opening the pressure sensitive nozzle valves.

The command for the spraying phase can be given manually by the user by means of a button inside the booth.

The ionisation apparatus can be of every kind. As a further example of ionisation apparatus is a so called ion accelerator which thanks to the natural magnetism of rare earth generating a magnetic field of about 9000 gauss causes the polarisation of the particles. This effect is enhanced by the circulation of the fluid several times through the ion accelerator. The high magnetic field furthermore has also an important sterilisation effect.

Relating to FIG. 26, a portable device is shown in which a praying gun is provided. The spraying gun 110 can be of the kind known for spraying paint, particularly having an tank on which the gun is mounted having an integrated suction pump. The said suction pump being electrically driven and communication with the suction pipe with the tank and with it output with a nozzle. Different nozzles 5 may be provided that can be alternatively mounted on the gun output. In combination therewith one or more stencils 111 having differently shaped patterns are provided. Thus it is possible to locally spray a tanning liquid and giving to the sprayed zone a special shape provided on the stencil. This allows to obtain tattoo like designs or patterns on the body of a user. The above mentioned combination of parts can form a kit for applying painted or tanning patterns on the body which can be housed in a bag.

The apparatus according to the invention may be further enhanced with different facilities. For example a device for displaying films or television programs can be provided and also a film can be displayed instructing the use of the apparatus, which film may be stored on a magnetic band, or in the form of a digital film on a digital storage medium.

Also means for giving vocal commands and emitting vocal instructions can be provided.

Means for automatic distribution of under wear or the like can be provided.

As already disclosed the apparatus may be provided with one or more further circuits for spraying different fluids or liquids. Air can be sprayed for drying and water or other liquids can be sprayed for washing.

The operative devices like the pumps, the tanks, the ionisator units, the electronic and electric units, the electro valves and the other additional devices can be placed on the trolley as illustrated in FIG. 23 so that it is very simple to change the bottles 80 and/or to carry out inspections or repair interventions.

What is claimed is:

1. An apparatus for painting a coating paint product, the apparatus comprising:
at least one column-shaped member;
at least one spray nozzle fitted on the at least one column-shaped member;
at least one tank containing coating paint product;
an electrically insulated, shower tray-like platform; and
at least one wall containing a jet-spraying nozzle, the at least one wall facing the at least one column-shaped member.

2. The apparatus as claimed in claim 1, wherein said product is vaporized and/or micronized and/or atomized by said spray nozzle placed on at least one column-like member, inside the booth.

3. The apparatus as claimed in claim 1, wherein that said nozzles are in a number of two or more.

4. The apparatus as claimed in claim 1, wherein said nozzles are preferably in a number of three to five.

5. The apparatus as claimed in claim 1, wherein said nozzles are high-pressure pneumatic atomizers.

6. The apparatus as claimed in claim 1, wherein said walls may be in a number of two or more enclosing completely or partially a user housing volume, while the nozzles are oriented essentially in the direction of a central zone of the said volume or a central column coaxial to the said volume.

7. The apparatus as claimed in claim 1, wherein said walls are curved about a vertical axis perpendicular to the booth base.

8. The apparatus as claimed in claim 1, wherein said walls are concave toward the inside of the booth.

9. The apparatus as claimed in claim 1, wherein said walls are translucent.

10. The apparatus as claimed in claim 1, wherein said platform rotates in a clockwise and/or counter-clockwise direction.

11. The apparatus as claimed in claim 1, wherein said platform is driven by a pneumatic motor.

12. The apparatus as claimed in claim 1, wherein said platform is electrically shielded by a connection to the ground potential.

13. The apparatus as claimed in claim 1, wherein said platform has the shape of a tray.

14. The apparatus as claimed in claim 1, wherein the paint product in use may be a tanning liquid and/or sea water and/or thermal spring water and/or thermal muds.

15. The apparatus as claimed in claim 1, wherein the paint product in use is sprayed in amounts of 1 ml to 200 ml.

16. The apparatus as claimed in claim 1, wherein the paint product in use is sprayed in the preferred amount of 80 ml.

17. The apparatus as claimed in claim 1, wherein the paint product in use may be electrostatically charged.

18. The apparatus as claimed in claim 1, wherein said liquid is charged electrostatically inside the tank/s that contain the liquid.

19. The apparatus claimed in claim 1, wherein an electrode and/or a sparking plug and/or a polarizing diode are associated to said tank and dip in said liquid or are in electric contact with the liquid.

20. The apparatus as claimed in claim 1, wherein said liquid is charged electrostatically while it passes through the pipe that connects the tank and the discharge nozzle/s.

21. The apparatus as claimed in claim 1, wherein the liquid is charged by radiative ionization.

22. The apparatus as claimed in claim 1, wherein the liquid is charged electrostatically while it passes through a transparent tube for connection of the tank and the spray nozzle/s, which transparent tube is adjacent to a UV lamp.

23. The apparatus as claimed in claim 1, wherein the liquid is atomized at the spry nozzles to liquid particles having an average diameter of about 20 μm.

24. The apparatus as claimed in claim 23, wherein the liquid is charged electrostatically with a positive charge as an effect of the previous atomization.

25. The apparatus as claimed in claim 1, wherein it includes a tubular inverted U-shaped upright 2 having nozzles in at least one of its stems.

26. The apparatus as claimed in claim 1, wherein it includes a lo tubular inverted U-shaped upright 2 providing the function of supporting the curvilinear walls.

27. The apparatus according to claim 1, further comprising a tray like platform (3) and a plurality of nozzles (5) which are placed at different heights above the platform and at different angular positions on an ideal surface enveloping a volume vertically aligned with the said platform (3), the nozzles being fed with a treatment liquid under pressure, the liquid being subjected to ionization or electrostatic charging before being fed to the nozzles and/or at the nozzles and/or immediately after leaving the nozzles by means of subjecting the liquid to the action of electric, magnetic, electromagnetic and/or mechanic energy.

28. The apparatus according to claim 27, wherein the nozzles (5) are distributed on a rotational symmetric enveloping surface around a central vertical axis of the platform (3).

29. The apparatus according to claim 27, wherein several vertical rows of nozzles are provided which are aligned along vertical axis at different positions on the ideal enveloping surface.

30. The apparatus according to claim 27, wherein the nozzles (5) are positioned on each row in a non uniform way, the nozzles t5) of a row at a certain height being positioned closer than the nozzles (5) at different s height along the row.

31. The apparatus according to claim 27, wherein the nozzles (5) are positioned on the enveloping surface vertically displaced the one with respect to the laterally adjacent forming spraying cones only slightly overlapping each other or not overlapping at all which cones are positioned in quincunx relationship.

32. The apparatus according to claim 31, wherein each row of nozzles t5) is vertically displaced with respect to the nozzles of the laterally adjacent row the cones of one row being placed in a quincunx relationship relatively to the cones of the laterally adjacent row.

33. The apparatus according to claim 27, wherein the enveloping ideal surface is a rectangular or elliptic surface or a polygonal surface being coaxial or concentric to a corresponding rectangular or elliptic or polygonal surface approximating the shape of the external surface of an upstanding body placed at the central zone of the platform (3).

34. The apparatus according to claim 27, wherein lateral walls (1) are provided departing vertically from the platform (3) and extending along the ideal enveloping surface.

35. The apparatus according to claim 27, wherein the nozzles (5) are carried by the said walls (1).

36. The apparatus according to claim 27, wherein feeding pipes (102) are provided connecting each nozzle s (5) with a feeding circuit (90) for the liquid to be sprayed, the feeding pipes (102) supporting the nozzles (5) and being housed with the nozzles (5) in a hollow space between an internal layer (201) and an external layer (101) forming the lateral wall (1).

37. The apparatus according to claim 35, wherein vertical or upright feeding pipes (102) are provided for each row of nozzles (5), each vertical pipe.(102) carrying the nozzles (5) of each vertical row and forming a liquids distributing chamber.

38. The apparatus according to claim 37, wherein the vertical or upright pipes (102) are distributed at different angular positions along the enveloping surface and are provided into two groups, the vertical pipes (102) of one group being provided on one side of the enveloping surface and the second group on the opposite side of the said enveloping surface relatively to a central or diametric section plane of the said enveloping surface along the larger diameter or the larger center line.

39. The apparatus according to claim 38, wherein the vertical or upright pipes (102) are placed in a symmetric way with respect to the said central or diametric section plane of the said enveloping surface along the larger diameter or the larger center line.

40. The apparatus according to claim 27, wherein at least a certain number of a second kind of nozzles is provided for spraying a different gas or liquid at least a further feeding circuit being provided for the said other liquid or gas.

41. The apparatus according to claim 40, wherein the other liquid is water.

42. The apparatus according to claim 40, wherein the other fluid is air.

43. The apparatus according to claim 27, wherein the platform (3) is shaped basin like and has a concave bottom degrading towards an output opening, the said output opening being connected to a collecting tank a pump (43) being provided for feeding the rest liquid accumulated in the platform (3) after spraying from the said platform (3) to the said tank (41).

44. The apparatus according to claim 27, wherein vent or air suction means (42) are provided sucking the air from the volume enclosed by the lateral wall (1) and feeding the said air to a liquid condenser, the said condenser having an output connected to the tank (41) directly or by means of a suction pump (40).

45. The apparatus according to claim 27, wherein the tank for the liquid to be sprayed is formed by the bottles (80) in which the liquid is sold or delivered which tanks are provided with an output sleeve (280), a closure cap (81) of the said bottle (80) being provided having mechanical clamping means (181) engaging the said output sleeve (280) and sealing means (82) for sealingly connecting the closure cap (81) and the output sleeve (280), which clamping means can be disengaged form the output sleeve (280) and which closure cap (81) carries a suction tube (83) sealingly passing through the said closure cap and reaching almost to the bottom of the bottle (80), the said suction tube (83) being connected to the feeding circuit of the liquid to the nozzles (5).

46. The apparatus according to claim 45, wherein the closure cap (81) carries a liquid level measuring or sensing means (84, 85).

47. The apparatus according to claim 45, wherein the collecting tank (41) is lo formed by an empty liquid bottle (80) a closure cap (81) being provided the suction tube (83) of which in connected at the rest liquid discharging circuit or pump and maximum level sensing means (85) being provided on the said closure cap (81).

48. The apparatus according to claim 27, wherein a certain number of nozzles aligned on at least one plane parallel to the platform (3) is carried by a bracket (60), the said bracket (60) being carried in a vertical slidable way on at least one vertical guide and actuator means being provided for displacing the said bracket up and down along the vertical guides, the bracket being formed by an open annular element which shape is correspondent to the enveloping surface.

49. The apparatus according to claim 48, wherein the bracket (60) is housed in the hollow space between the internal layer (201) and the external layer (101) forming the lateral wall (1), the said hollow space having a concentric or coaxial spare with respect to the shape of the bracket (60), while the internal layer (201) of the lateral wall (1) has vertical openings coinciding with the vertical path of each nozzle (5) on the bracket (60).

50. The apparatus according to claim 49, wherein the nozzles (5) on the bracket forms blade like spaying jets.

51. The apparatus according to claim 27, wherein the nozzles (5) are of the kind mixing the liquid to be sprayed with gas or air under pressure for atomization of the said liquid separate feeding circuits (66, 67) being provided for the gas or air under pressure and for the liquid.

52. The apparatus according to claim 51, wherein the gas and the air feeding circuits (66, 67) are provided with valves for closing alternatively one for both the said feeding circuits, thereby causing the nozzles(5) to eject only liquid or only gas.

53. The apparatus according to claim 48, wherein further nozzles for a further different gas or liquid are provided which are connected to a dedicated feeding circuit.

54. The apparatus according to claim 27, wherein the nozzles (5) are connected to a circulating circuit having a feeding branch (90) of the liquid and a return branch (91) of the liquid. The feeding branch being connected to a first circulating pump (92), the feeding branch being connected to the feeding output of the pump (92), the return branch (91) being connected to the suction of the pump (92), in the return branch (91) being provided a device (93) for ionizing the liquid and a valve (94) for closing the return branch (91), the suction of the pump (92) being further connected to a liquid tank (80), the pump (92) being capable of generating a variable liquid pressure within the circulating circuit, control means being provided for driving the pump at a range for generating a high pressure in the liquid and closing the return branch (91) by means of s the valve (94) and for driving the pump at a range generating low pressure in the liquid and activating the ionisator (93) and opening the valve (94) of the return branch (91).

55. The apparatus according to claim 54, wherein two pumps are provided a first pump (92) being a low pressure pump and being activated for feeding the liquid from the tank (80) to the circuit and circulating the said liquid within the feeding and the return branch (90, .91) while the valve (94) in the return branch (91) is open and the ionisator is activated and a second pump (95) for generating a high pressure at least in the feeding branch (90), which second high pressure pump (95) is provided in the feeding branch after the low pressure pump (92) and is activated for generating the spraying pressure when the valve (94) on the return branch is closed, the second pump having a by-pass branch or being open for the liquid flow when inactive.

56. The apparatus according to claim 55, wherein the low pressure pump (92) is a self triggering pump controlled by the liquid level sensor within the tank (80).

57. The apparatus according to claim 55, wherein the second pump is driven by a brushless motor by means of an inverter (97) controlled by a central processing unit (CPU) (98), the said pump being able to be driven at different rotation speeds for generating different values of the liquid pressure.

58. The apparatus according to claim 55, wherein the CPU controls the valves (94) in the return branch (91) which is an electrovalve and the ionisator and has s timing means counting time periods.

59. The apparatus according to claim 27, wherein a program is provided for driving the low pressure pump (92) together with the ionisator for a given period of time and for activating the high pressure pump (95) at different rotational speeds corresponding to at least two different liquid pressures, and closing or opening the valve (94) in the return branch (91), the said program providing different operational phases:
a first circuit loading phase consisting in activating the low pressure pump (92) for sucking the liquid from the tank (80) into the feeding branch (90) and for opening the valve (94) in the return branch (91);
a second stand-by phase in which low pressure pump (92) is activated for providing circulation of the liquid through the ionization means (93) which are activated, the said phase being carried out for a period of time sufficient long for ensuring continuous mixing and ionization of a certain amount of the liquid;
a third ready to start phase during which the high pressure pump is activated at a low level rotation speed so that the pressure does not increase as high as to open generate a liquid output at the nozzles but the pressure in the system reaches a higher pressure from which the optimum spraying pressure can be rapidly reached; and
a fourth spraying phase in which the high pressure pump is brought to a higher rotation rate such as to allow the optimum spraying pressure to be reached and the electro valve in the return branch (91) is closed.

60. The apparatus according to claim 27, wherein manual means are provided for starting the spraying phase when the circuit is in a ready to start phase.

61. The apparatus according to claim 27, wherein pressure sensitive nozzle-valves are provided which open the connection of the nozzles (5) to the feeding Circuit only when the liquid in the feeding circuit reaches certain pressure.

62. The apparatus according to claim 61, wherein each nozzle is provided at a peripheral wall of a feeding chamber (205), the said feeding chamber being connected to the feeding branch (90) by means of a pressure sensitive valve (305, 405, 505).

63. The apparatus according to claim 62, wherein the pressure sensitive valve being formed by a piston like shutter (505) slidably displaceable within the feeding chamber (205) from a position in which it closes the input opening (305) of the feeding chamber in a position at which the said input opening (305) is not closed, elastic means being provided urging the said piston like shutter in the position closing the input opening (305).

64. The apparatus according to claim 63, wherein said feeding chamber (205) and the pressure sensitive valve (305, 405, 505) housed therewith are integrated in a nozzle body, each nozzle (5) being provided with it own pressure sensitive valve.

65. The apparatus according to claim 59, wherein during the ready to start phase the liquid pressure in the feeding branch (90) is lower than the pressure needed s to open the pressure sensitive valves of the nozzles (5), while during the spraying phase the pressure of the liquid is higher than the pressure needed to open the pressure sensitive valves of the nozzles (5).

66. The apparatus according to claim 27, wherein at least part of the pump or pumps and/or of the valves and/or of the driving units and/or of the pipes forming the feeding and/or return branches (90, 91) and/or of the tanks (80, 40) and/or of the control units is housed on a trolley like member which is housed in a lateral extension room of a booth formed by the lateral wall (1) and the platform (3), the said lateral extension room being provided with a door and the said trolley being displaceable inside and outside the said extension room, while flexible pipe and or cable connections are provided to the circuit parts and/or to the electric connection cables residently and fixedly mounted on the lateral wall (1) and or on the platform (3).

67. The apparatus according to claim 27, wherein the high pressure pump is drive in order to reach a spraying pressure from 30 to 100 bars, preferably from 60 to 70 bar.

68. The apparatus according to claim 27, wherein each spraying cycle lasts several seconds particularly from 1 to 3 seconds.

69. The apparatus according to claim 27, wherein between two spraying cycles a rest cycle of predetermined duration is provided which is sufficient for a certain amount of s the vaporized or atomized liquid particles in the air to fall down by gravity.

70. The apparatus according to claim 27, wherein the nozzles and the pressure of the liquid are defined in such a way as to generate drops having a range between 5 and 40 microns and to avoid turbulences.

71. (canceled)

72. An apparatus for painting a coating paint product and a tanning liquid, the apparatus comprising a spraying gun, the spraying gun comprising:
a handle;
an electric pump integrated in the handle;
means for securing the spraying gun to a liquid tank;
a suction sleeve connected to the spraying gun and housed in the liquid tank;
a spraying nozzle (5) at the pump output; and
an activation button integrated in the handle.

73. The apparatus according to claim 72, further comprising one or more stencils (11) having different patterns.

74. The apparatus according to claim 72, wherein several nozzles (5) of different kinds are provided, the several nozzles being suitable for mounting on the spraying gun.

75. (canceled)

76. The apparatus according to claim 72, wherein the apparatus is provided as a kit for providing a human body with a tattoo like pattern.

77. A method for painting a coating liquid using nozzles, the method comprising the steps of:
electrically charging the liquid;
feeding the liquid to the nozzles;
atomizing the electrically charged liquid; and spraying the liquid against a body;
the energy field for electrically charging the liquid being confined to the outside the body.

78. The method according to claim 77, wherein the energy field comprises one or more of an electric field, a magnetic field, and an electromagnetic field.

79. The method according to claim 77, wherein the liquid is electrically charged by mechanical energy.

80. The method according to claim 77, wherein the liquid is electrically charged and/or ionized by reducing the droplets size of the vaporized or atomized liquid to a size where electrostatic charge inhomogenities of the molecular structure of the liquid becomes relevant.

81. The method according to claim 77, wherein the liquid is atomized or vaporized to a droplet size from 5 to 40 microns.

82. The method according to claim 77, wherein spray of the liquid is carried out from several directions against the body to be treated and at several height of the said body by providing several spray jets distributed over a body enveloping surface at different heights thereof.

83. The method according to claim 77, wherein the spray jets are oriented in such a way one with respect to the other that at a certain distance the spraying cones generated do only slightly overlap or do not overlap at all.

84. The method according to claim 83, wherein the spray jets are oriented in such a way as to generate sprayed areas on the body to be treated which have a quincunx arrangement.

85. The method according to claim 77, wherein the spray jets are provided closer to one another at certain zones of the body to be treated.

86. The method according to claim 77, wherein each spray jet is originated approximately from the same distance from the corresponding target zone of the body to be treated.

87. The method according to claim 77, wherein groups of spray jets are generated, the jest of each group being positioned on the enveloping surface on a vertical row of jets and the different vertical row of jets being distributed at different angular positions along an enveloping surface of the body to be treated.

88. The method according to claim 77, wherein the enveloping ideal surface is a rectangular or elliptic surface or a polygonal surface being coaxial or concentric to a corresponding rectangular or elliptic or polygonal surface approximating the shape of the external surface of an upstanding body.

89. The method according to claim 77, wherein a plurality of spray jets is generated along a line on the enveloping surface, the said jets being displaced along the axial dimension of the enveloping surface in both directions.

90. The method according to claim 89, wherein the speed of displacement is different at different parts of the path of the jets.

91. The method according to claim 77 wherein the spray jets are generated by providing the liquid under trig pressure conditions and feeding the said high pressure liquid to one or more spray nozzles (5), the spray nozzles (5) being open to input of the high pressure liquid only starting for a certain liquid pressure.

92. The method according to claim 77, wherein one or more stencils are provided for cooperating with one or more spray jets.

93. The method of claim 77, wherein:
a single spray jet of the liquid is provided, the single spray jet having an aperture cone for treating only a local zone of the body; and
the liquid is delivered through one or more stencils suitable for providing a pattern on the body.

94.-95. (canceled)
